(19) United States
(12) Patent Application Publication
(10) Pub. No.: US 2016/0338884 A1
(43) Pub. Date: Nov. 24, 2016

(54) ODOR CONTROL IN PERSONAL CARE PRODUCTS

(71) Applicant: KIMBERLY-CLARK WORLDWIDE, INC., Neenah, WI (US)

(72) Inventors: Roger Bradshaw Quincy, Cumming, GA (US); John Gavin MacDonald, Decatur, GA (US); Anthony Stephen Spencer, Woodstock, GA (US)

(73) Assignee: Kimberly-Clark Worldwide, Inc., Neenah, WI (US)

(21) Appl. No.: 15/107,009

(22) PCT Filed: Dec. 26, 2014
(86) PCT No.: PCT/US14/72436

§ 371 Date: Jun. 21, 2016

Related U.S. Application Data
(60) Provisional application No. 61/921,240, filed on Dec. 27, 2013.

(51) Int. Cl. A61F 13/84; A61F 13/496; A61L 15/18; A61F 13/49; A61L 9/014; A61L 15/46; A61F 13/534; A61F 13/472

(52) U.S. Cl. CPC A61F 13/8405; A61F 13/534; A61F 13/496; A61F 13/472; A61F 13/49009; A61L 9/014; A61L 15/46; A61L 15/18; A61F 2013/5349; A61F 2013/8408; A61L 2300/404; A61L 2300/104; A61L 2400/12; A61F 2013/842

(57) ABSTRACT

A urine odor control system for personal care products includes a substrate with a formulation of activated carbon and an absorbent core with a formulation of silver. The personal care product includes diapers, training pants, absorbent underpants, adult incontinence products, or feminine hygiene products. The personal care product can include a liquid-permeable bodyside liner, liquid-impermeable outer cover affixed to the bodyside liner, wherein the absorbent core and substrate are disposed between the bodyside liner and the outer cover.

### CROSS-REFERENCE TO RELATED APPLICATIONS

This application claims priority to U.S. Provisional Application 61/921,240, filed on Dec. 27, 2013. U.S. Provisional Application 61/921,240 is incorporated herein in its entirety.

### BACKGROUND

The present disclosure concerns processes and products for the alleviation and control of urine odors in personal care products.

Disposable personal care products perform a needed function in today's busy society, freeing caregivers and users from the chore of washing reusable products and allowing for the quick and easy disposal of body wastes. As leakage issues have been reduced because of improved designs, the control of odors has become more important to the consumer. This is a particular concern to users of incontinence and feminine hygiene products.

Odor is often used by consumers as a signal that a personal care product should be changed. The detection of the odor depends, however, on the acuity of the sense of smell of the consumer, an acuity that often declines with age. Relying on the odor of the product also means that the odor may become offensive before the product is changed, an unacceptable signal.

Many technologies have been evaluated in an attempt to reduce the odors that emanate from these products during use. For example, incontinence pad liners with an activated carbon ink (with binder) printed thereon was recently introduced. Many urine odor ranking panel (ORP) studies have shown improvements in odor for the carbon treated liner. However, complete elimination of odor has not been achieved with activated carbon.

In another example, a surge layer of an incontinence pad was treated with activated carbon and/or silver nanoparticles. However, complete elimination of urine odor has not been achieved with activated carbon and/or silver in the surge material.

It is also important that anything added to a personal care product to reduce odor should be safe and remain in place without migrating through the product, as has occurred in previous attempts to address this matter. Absorbent and/or adsorbent (sorbent) particles, for example, should not escape from the product nor be noticeable to the consumer. It is also desirable that the silver particles do not escape and be in contact with the skin, potentially disrupting the skin microflora or developing coloration to the skin.

It is clear that there exists a need for an article that allows for the control of odors due to bodily wastes in personal care products.

### SUMMARY

In response to the foregoing difficulties encountered by those of skill in the art, a personal care product is disclosed. Research leveraging the unique synergistic benefit of activated carbon with silver led to the discovery of where to apply these actives to deliver urine odor elimination in the most efficacious manner. In one aspect the personal care product includes a substrate with activated carbon incorporated therein, and an absorbent core with silver particles incorporated therein. The personal care products include diapers, training pants, absorbent underpants, adult incontinence products, and feminine hygiene products.

In another aspect, there is a method for reducing urine odor in a personal care product to a level below human detection. The method includes the steps of disposing a substrate comprising activated carbon onto an absorbent core comprising silver particles, and disposing the paired substrate and absorbent core between an outer cover and a bodyside liner.

Other features and aspects of the present disclosure are described in more detail below.

### BRIEF DESCRIPTION OF THE DRAWINGS

The present disclosure will be more fully understood, and further features will become apparent, when reference is made to the following detailed description and the accompanying drawings. The drawings are merely representative and are not intended to limit the scope of the claims.

FIG. 1 is a drawing of a feminine hygiene product.

FIG. 2 is a drawing of an adult incontinence product.

FIG. 3 is a drawing of a cross-section of an adult incontinence product.

FIG. 4 is a drawing of an absorbent underpant.

FIGS. 5 through 10 are charts illustrating the results of odor testing with different urine-odor eliminating substances.

Repeat use of reference characters in the present specification and drawings is intended to represent the same or analogous features or elements of the present disclosure. The drawings are representational and are not necessarily drawn to scale. Certain proportions thereof might be exaggerated, while others might be minimized.

### DETAILED DESCRIPTION OF THE DISCLOSURE

The present disclosure involves the control of odors in absorbent articles. As used herein, an “absorbent article” refers to any article capable of absorbing water or other fluids. Examples of some absorbent articles include, but are not limited to, personal care absorbent articles, such as diapers, training pants, absorbent underpants, adult incontinence products, feminine hygiene products (e.g., sanitary napkins), swim wear, baby wipes, and so forth; other absorbent articles, such as food service wipers; clothing articles; and so forth. Materials and processes suitable for forming such absorbent articles are well known to those skilled in the art.

The control of urine dors in personal care products is of particular interest to adults that wear feminine hygiene pads and/or incontinence products. The desire to avoid embarrassment and discovery due to unpleasant odors is important to consumers of these products.

The treatment combination that reduces urine odor to level that are below human detection involves two principle components: activated carbon and nano- or micro-particle silver. These two principle components are placed in separate locations in the disposable product to accommodate ease of manufacture and unfettered access to each odor elimination substance.

The combination of the activated carbon and silver components described herein has been found to have an unexpected and unique synergistic effect in eliminating odors. The combination provides a significantly larger odor reduction that can be achieved by either component alone, even at relatively high concentrations. The combination also provides greater odor reduction than can be achieved by treating the absorbent article with an antimicrobial alone or a single odor sorbent.

### Absorbent Articles

Disposable absorbent incontinence products are designed to be removed and discarded after a single use. By single use it is meant that the disposable absorbent incontinence product will be disposed of after being used once instead of being laundered or cleaned for reuse, as is typical of regular cloth underwear. Examples of some commercially available disposable absorbent incontinence products include diapers, training pants, pads, pantyliners, fitted briefs, belted shields, guards for men, protective underwear, and adjustable underwear.

In one particular aspect of the disclosure, an absorbent article includes components such as: a liquid-permeable layer (e.g., body-side liner, surge layer, etc.), a liquid-impermeable layer which might have moisture vapor permeability or breathability (e.g., outer cover, ventilation layer, baffle, etc.), an absorbent core, an elastic member, and so forth. Several examples of such absorbent articles are described in U.S. Pat. No. 5,197,959 to Buell; U.S. Pat. No. 5,085,654 to Buell; U.S. Pat. No. 5,634,916 to Lavon, et al.; U.S. Pat. No. 5,569,234 to Buell, et al.; U.S. Pat. No. 5,716,349 to Taylor, et al.; U.S. Pat. No. 4,950,264 to Osborn, III; U.S. Pat. No. 5,009,653 to Osborn, III; U.S. Pat. No. 5,509,914 to Osborn, III; U.S. Pat. No. 5,649,916 to DiPalma, et al.; U.S. Pat. No. 5,267,992 to Van Tillburg; U.S. Pat. No. 4,687,478 to Van Tillburg; U.S. Pat. No. 4,285,343 to McNair; U.S. Pat. No. 4,608,047 to Mattingly; U.S. Pat. No. 5,342,342 to Kitaoka; U.S. Pat. No. 5,190,563 to Herron, et al.; U.S. Pat. No. 5,702,378 to Widlund, et al.; U.S. Pat. No. 5,308,346 to Sneller, et al.; U.S. Pat. No. 6,110,158 to Kielpikowski; U.S. Pat. No. 6,663,611 to Blaney, et al.; and WO 99/00093 to Patterson, et al.; each of which are incorporated herein in their entirety to the extent they do not conflict herewith.

Many of the disposable absorbent incontinence underwear are similar in appearance, size, and shape to regular cloth underwear except that they are formed from a variety of different materials including absorbent and elastic materials. The absorbent materials allow the disposable absorbent incontinence underwear to absorb and retain body waste while the elastic material permits the disposable absorbent incontinence underwear to snugly conform to the anatomy of the wearer's torso.

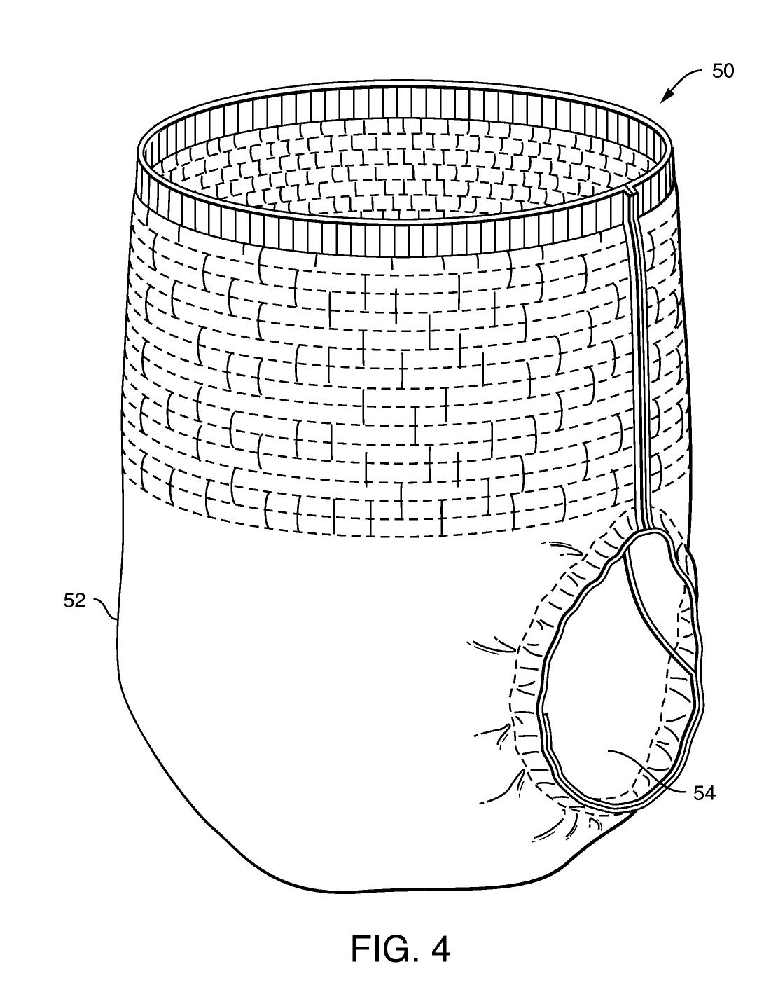

Much of the disposable absorbent incontinence underwear sold today has a unitary configuration that is similar to regular cloth underwear in that the disposable absorbent incontinence underwear is constructed with a waist opening and a pair of leg openings and needs to be pulled onto the body like normal underwear. For example, absorbent underpant 50 as shown in FIG. 4 has an outer cover or baffle 52, a body-side liner 54, and an absorbent core (not shown). Further discussion regarding absorbent underpants can be found, for example, in U.S. Pat. No. 6,240,569 to Van Gompel; U.S. Pat. No. 6,367,089 to Van Gompel; and U.S. Patent Publication No. 2004/0210205 A1 to Van Gompel et al., which are incorporated herein in their entirety by reference thereto to the extent they do not conflict herewith.

Other disposable absorbent incontinence underwear has an open configuration. By an open configuration it is meant that the disposable absorbent incontinence underwear does not have a waist opening and a pair of leg openings before it is positioned about the wearer's torso. Typically, disposable absorbent incontinence underwear having an open configuration has a relatively flat or convex shape before it is secured around the torso of the wearer. Commonly, disposable absorbent incontinence underwear having an open configuration has an approximately rectangular or hourglass shape. Such products are described in U.S. Pat. No. 4,500,316 to Damico, which is incorporated herein in its entirety by reference thereto to the extent it does not conflict herewith.

A belted shield is still another type of a disposable absorbent incontinence product that has an open configuration and is held about the wearer's torso by a belt or a pair of straps, as described in U.S. Pat. No. 5,386,595 to Kuen et al. and U.S. Pat. No. 4,886,512 to Damico et al., which are incorporated herein in their entirety by reference thereto to the extent they do not conflict herewith.

Another type of incontinence product is a guard for men, which resembles an absorbent pad that can conform to the male genitalia, as described in U.S. Pat. No. 5,558,659 to Sherrod et al., which is incorporated herein in its entirety by reference thereto to the extent it does not conflict herewith.

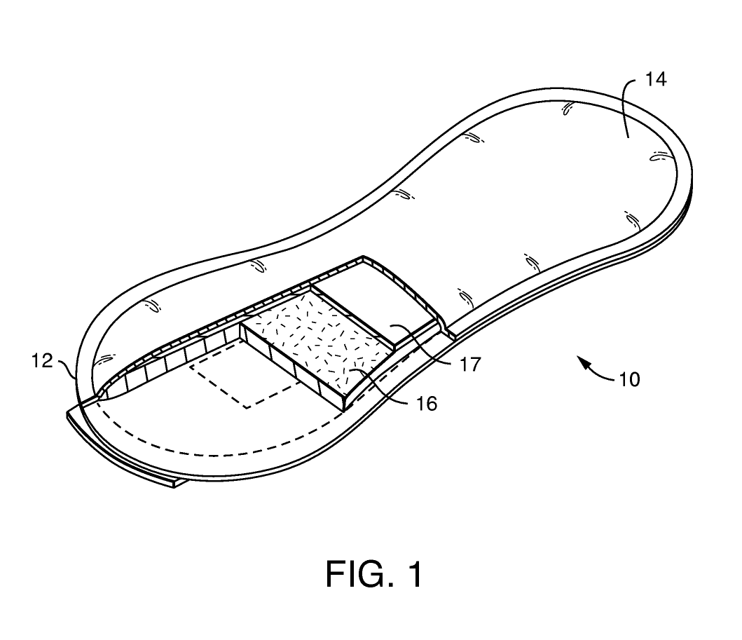

While not ideal, some women wear feminine hygiene pads to absorb urine, such as the pad 10 shown in FIG. 1. Pad 10 includes a body-side liner 14, which extends to a pad perimeter 12. Underneath liner 14 is a tissue layer 17 and an absorbent core 16. The tissue may be substituted by a different substrate, such as a non-woven material.

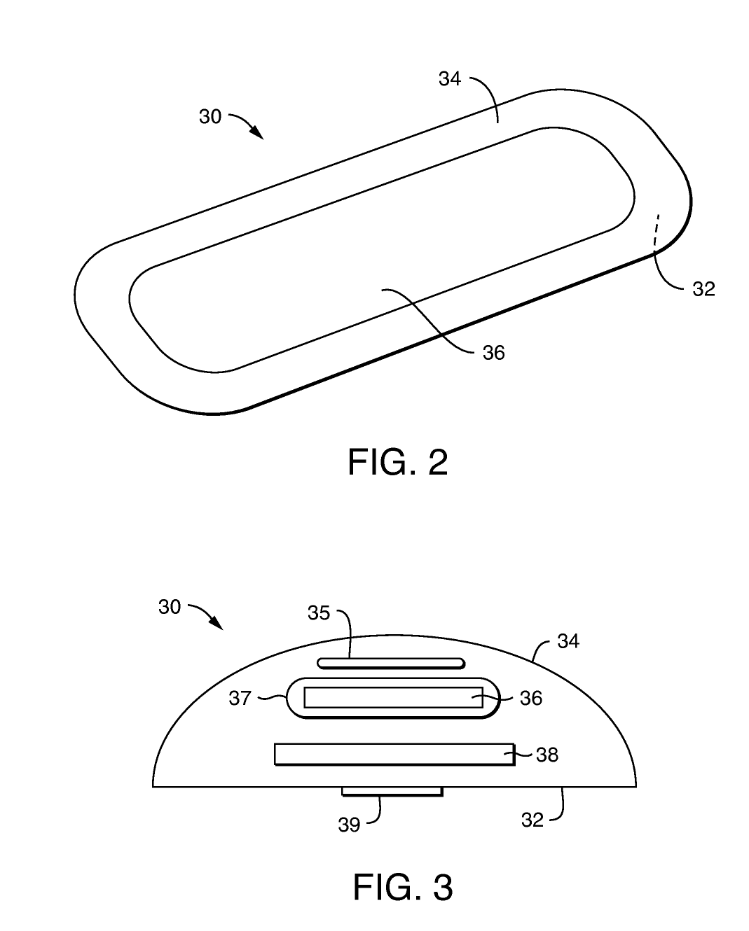

Like feminine hygiene pads, feminine incontinence pads 30 as shown in FIGS. 2 and 3 have a baffle or outer cover 32, a body-side liner 34, and various layers in between, which includes an absorbent core 36. The absorbent core 36 has a body-facing surface adjacent the body-side liner 34, a garment-facing surface adjacent the outer-cover 32, and a pair of longitudinal sides. FIG. 3 is a cross-section of one non-limiting example of incontinence product. The body-side liner 34 is at the top of FIG. 3. The body-side liner 34 is designed to allow body fluid, particularly urine, to quickly pass through and be received by an absorbent core 36. The bodyside liner 34 is placed in contact with the genital area of a human body. A surge layer 35 is positioned below the liner 34. The surge layer 35 acts as a reservoir to accept large surges of liquid and slowly release them to the subsequent layers. Below the surge layer 35 is the absorbent core 36 surrounded by a substrate in the form of a tissue wrap 37. The absorbent core 36 typically includes fluff and superabsorbent particles. The superabsorbent particles are loose and very small and therefore can escape onto the body or clothing unless contained. The substrate or tissue wrap 37 serves to prevent superabsorbent and silver particles from migrating from the absorbent core to the user's skin. Under the substrate-wrapped absorbent core 36 is a baffle or outercover 32.

In one aspect of the disclosure, the substrate containing activated carbon is disposed onto the absorbent core 36 by wrapping it at least around the body-facing surface and longitudinal sides. A substrate such as tissue wrap 37 may be fully wrapped about the absorbent core 36 so that the garment-facing surface is covered as well. In another aspect of the disclosure, the substrate containing activated carbon is a strip that covers as much as 100 percent the surface area of the body-facing surface of the absorbent core 36. The substrate strip is disposed onto the body-facing or the garment-facing surface of the absorbent core 36. In yet another aspect of the disclosure, two substrate strips are used, one covering the body-facing surface of the absorbent core 36, and the other, the garment-facing surface of the absorbent core 36.

Many products also have an adhesive strip 39 to help hold the product in place during use by adhering it to the user's underclothes. Further, in one embodiment, there is an optional second absorbent layer, such as the airlaid layer 38 seen in FIG. 3. Airlaid layer 38 may be placed either below the tissue wrapped absorbent core 36 as shown, or above the tissue wrapped absorbent core 36.

Pads typically have a thickness of about 2.5 centimeters (cm) or less. Desirably, the thickness of a pad is less than about 1 cm. More desirably, the thickness of a pad is less than about 0.7 cm. A pad can have a length of from between about 15 cm to about 50 cm, and a width of from between about 2 cm to about 15 cm. Pads may have a rectangular, hourglass, or asymmetrical configuration.

A pantyliner, not shown, is a relatively thin absorbent pad having a thickness of about 1 cm or less. Desirably, the thickness of a pantyliner is less than about 0.5 cm. A pantyliner can have a length of from between about 15 cm to about 50 cm and a width of from between about 2 cm to about 15 cm. The pantyliner may have a rectangular, hourglass or asymmetrical configuration and may contain the same components as the pad shown in FIG. 3, or at least the body-side liner 34, the substrate such as tissue wrap 37, an absorbent core 36, and an outer cover 32.

In the present disclosure, the activated carbon is placed into the substrate such as tissue wrap 37 during the manufacture thereof. This ensures that the carbon particles are bound into the substrate and will not migrate to the article liner 34. In one embodiment of the disclosure, the substrate in the form of tissue wrap 37 is made using a conventional cellulose tissue manufacturing process except that activated carbon particles or fibers are added to the pulp slurry in a concentration sufficient to result in a tissue having an effective amount of activated carbon. Conventional tissue products are made according to widely known papermaking-type processes. For example, U.S. Pat. No. 5,129,988 to Farrington, Jr.; U.S. Pat. No. 5,772,845 to Farrington, Jr. et al.; and U.S. Pat. No. 5,494,554 to Edwards et al. each discloses various tissue-making methods, which are incorporated herein in their entirety by reference thereto to the extent it does not conflict herewith.

Research has shown that adding silver particles to a sheet containing activated-carbon actually reduces the odor-reduction capability of these active materials. Therefore, there is a benefit to keeping these two active materials separated from each other within the absorbent structure. Furthermore, it is desirable to include the silver particles in the absorbent core. This mitigates migration of the silver to the wearer's skin, and reduces the possibility of upsetting the skin microflora. The silver particles are added to the absorbent core during manufacture.

More information concerning incontinence products can be found, for example, in U.S. Pat. No. 6,921,393 to Tears et al., which is incorporated herein in its entirety by reference thereto to the extent it does not conflict herewith.

### Activated Carbon

Activated carbon, in particle or fiber form may be used. The activated carbon may be derived from wood, coconut husks or other materials. Desirably, the activated carbon has a great enough surface area and volume of meso-pores to facilitate rapid removal of odor compounds from urine. For instance, in one embodiment, the particle size may be below about 45 or 50 microns in size, the surface area about 1400-1800 m²/g, and the pore volume of about 1.1-1.3 cc/g. One suitable activated carbon is NUCHAR SA-20 from MWV Specialty Chemicals, North Charleston, S.C.

The amount of carbon present in the absorbent article is about 0.01 g to about 0.4 g, or about 0.03 g to about 0.3 g, or about 0.02 g to about 0.2 g. The carbon is present in the substrate such as tissue wrap 37.

### Silver

In one embodiment, the silver formulation may include silver microparticles such as MICROSILVER BG-TEC, MICROSILVER BG or MICROSILVER BG-MED available from Bio-Gate AG, Nurnberg, Germany.

In another embodiment, the silver formulation may be silver nanoparticles such as SILVAGARD available from AcryMed, Inc., Beaverton, Oreg., USA. According to AcryMed, Inc., SILVAGARD silver nanoparticles are formed chemically in a solution. The nanoparticles are uniform in size (about 10 nm) and because of proprietary technology they do not agglomerate to form large particles, but stay in suspension pending application to other materials. The SILVAGARD silver nanoparticle technology is further described in U.S. Patent Publication No. 2007/003603A1 to Karandikar et al., which is incorporated herein by reference to the extent it does not conflict herewith.

The silver formulation, regardless of the silver particle size, is a liquid formulation. The formulation can be either aqueous or solvent based, depending on the needs or characteristics of the substrate to be treated.

In one embodiment, after the silver formulation is prepared, the fluff component of the absorbent core 36 is contacted with the formulation by immersing, spraying, printing, or by any other suitable application means. In some aspects of the disclosure, the fluff component in the absorbent core 36 is uniformly treated so that there is almost no fluff present that is not associated with silver particles.

The amount of silver actually deposited is controlled by adjusting the silver concentration in the formulation and the treatment conditions that govern the amount of silver formulation that is deposited on the fluff substrate, referred to as “wet pickup”. For a spray application, treatment conditions will include type of spray nozzle, pressure of delivered spray, and dwell time of substrate in the spray zone. Desirably, the amount of silver deposited in the absorbent core is about 50 ppm to about 10,000 ppm, or about 0.0003 to 0.06 gram of silver. An AUTOJET 1550 Modular spray system (Spraying Systems Co., Wheaton, Ill.), with SPR-I110 spray cabinet was used to spray coat samples. The fluff is pre-treated with the silver material and dried before combining with the superabsorbent particles.

The amount of silver in the absorbent core is at least 0.0001 g, or may be about 0.0001 g to 0.05 g, or about 0.001 g to about 0.02 g.

### EXAMPLES

For each code described in the Examples 1 through 4, POISE incontinence pads, available from Kimberly-Clark Corp. of Dallas, Tex., USA, with the label “Moderate Absorbency” were purchased from a local store for a urine odor ranking panel (ORP) study. The absorbent core and tissue wrap were removed from the commercial pads and replaced with the following components to produce the various codes.

For each code in the Examples 1, 2 and 4, the activated carbon tissue layer was produced by adding activated carbon during the manufacturing process. The 21 gsm activated carbon tissue of Examples 1, 2 and 4 included 1 gsm of NUCHAR SA-20 activated carbon, available from MWV Specialty Chemicals, North Charleston, S.C. The tissue made in the manufacturing process had the following characteristics: weight 21 g/m²; thickness 75 μm; tensile strength 2800 cN/30 mm machine direction (“MD”) and 670 cN/30 mm cross-machine direction (“CD”); stretch 1.6% MD, 3.0% CD; and air permeability 6725 cm³/cm².min (1 kPa).

For each code described with activated carbon in the Example 3, the 20 gsm tissue was produced by adding 1 gsm of NUCHAR SA-20 activated carbon in a laboratory process.

### Example 1

Code ID 513 (Water Control): 10″ by 17″ absorbent-core handsheets were formed as follows. 30 40 grams of BASF HYSORB 8760AD superabsorbent particles were mixed with 48.8 grams of WEYERHAEUSER NB416 fluff in a vacuum former having a tissue-lined screen. The handsheets were densified to the desired thickness of about 6 mm by feeding them through two smooth calendered nip rolls at a pressure of about 400 psi.

An oval shaped die about 2.5″ wide by about 9.5″ long was used to cut absorbent core specimens from the handsheets. The oval-shaped die used to cut the handsheets was also used to cut the tissue. Die-cut tissue was aligned with and placed on both the body-facing surface and the opposite garment-facing surface of the absorbent core to form an absorbent body. The absorbent body was placed in the pad and stapled in place at one end of the pad.

Code ID 804 (Urine Control):

The absorbent core and tissue layers described for code ID 513 were also used for this code. As with code ID 513, the die-cut tissue was aligned with and placed on both the body-facing surface and the opposite garment-facing surface of the absorbent core to form an absorbent body. The absorbent body was placed in the pad and stapled in place at one end of the pad.

Code ID 365: Multiple 10″ by 17″ handsheets that contained 11.6 grams of WEYERHAEUSER NB416 fluff were made on a vacuum former having a tissue-lined screen. The handsheets with WEYERHAEUSER NB416 fluff were cut to 10″ by 13″ and then individually sprayed with an aqueous silver formulation, specifically, BIO-GATE ZSM-MICROSILVER that had been diluted with distilled water to contain 699 ppm silver. Each sheet was sprayed twice on each side using an 8001 spray nozzle at a cycle velocity of 630 (63 feet/min), resulting in a spray head pressure of 80 psi. The treated sheets were dried in an air drier for 4 minutes at 95° C. The wet pickup was 371%, indicating that there was about 2,592 ppm of silver on the sheet.

Next, 48.8 gram batches of the silver-treated fluff were mixed with 40.0 gram batches of BASF HYSORB 8760AD superabsorbent particles to make absorbent core handsheets on a vacuum former having a tissue-lined screen. Finally, the absorbent core handsheets were densified to the desired thickness of about 6 mm by feeding through two smooth calendered nip rolls at a pressure of about 400 psi.

An oval shaped die about 2.5″ wide by about 9.5″ long was used to cut absorbent core specimens from the silver-treated fluff and superabsorbent handsheets. The oval-shaped die was also used to cut tissue that contained activated carbon. The die-cut activated carbon tissue was aligned with and placed on both the body-facing surface and the opposite garment-facing surface of the absorbent core to form an absorbent body. The absorbent body was placed in the pad and stapled in place at one end of the pad.

Code ID 127: 10″ by 17″ absorbent-core handsheets were formed as follows. 40.0 g of BASF HYSORB 8760AD superabsorbent particles were mixed with 46.4 g of WEYERHAEUSER NB416 fluff and 2.4 grams of activated carbon (CALGON OL 20×50) in a vacuum former having a tissue-lined screen. Next, the handsheets were densified to the desired thickness of about 6 mm by feeding through two smooth calendered nip rolls at a pressure of about 400 psi.

10″×14″ sheets of 20 gsm plain tissue (without carbon) were individually sprayed with an aqueous silver formulation, specifically, BIO-GATE ZSM-MICROSILVER that had been diluted with distilled water to contain 7410 ppm silver. Each sheet was sprayed twice on one side using an 8001 spray nozzle at a cycle velocity of 630 (63 feet/min), resulting in a spray head pressure of 80 psi. The treated sheets were dried in an air drier for 3 minutes at 100° C. The wet pickup was 929%, indicating that there was about 69,000 ppm of silver on the sheet.

An oval shaped die about 2.5″ wide by about 9.5″ long was used to cut absorbent core specimens from the absorbent-core handsheets that contained activated carbon. The oval-shaped die was also used to cut the silver-treated tissue. The die-cut tissue was aligned with and placed on both the body-facing surface and the opposite garment-facing surface of the absorbent core to form an absorbent body. The absorbent body was placed in the pad and stapled in place at one end of the pad.

The four codes of Example 1 are summarized in Table 1.

TABLE 1

| Code ID | Codes | Components |
|---|---|---|
| 513 | POISE control (water insult) | POISE moderate absorbency pad - untreated |
| 804 | POISE control (urine insult) | POISE moderate absorbency pad - untreated |
| 365 | MWV NUCHAR SA-20 | POISE moderate absorbency pad with activated carbon |
| | Activated Carbon and BIO- | treated tissue (1 gsm => 0.03 g carbon in product) and |
| | GATE ZSM MICROSILVER | with silver treated fluff (2592 ppm Ag; => 0.016 g Ag in |
| | silver particles | product) |
| 127 | CALGON 20 × 50 Activated | POISE moderate absorbency pad with activated carbon |
| | Carbon and BIO-GATE | treated fluff (at 5% => 0.31 g carbon in product) and with |
| | ZSM MICROSILVER silver | silver treated tissue wrap (68,859 ppm Ag; => 0.041 g Ag |
| | particles | in product) |

There were 10 pads per Code ID evaluated in the Odor Ranking Panel test.

Human urine was collected, pooled, filter sterilized, and then inoculated with bacteria (Proteus mirabilis, Klebsiella pnuemonae, E. faecalis, and E. coli). A fixed amount of urine (78 ml) was placed on each pad and the pad was incubated at 37° C. for four hours. Ten trained panelists were then exposed to each of the codes and asked to rank them for total odor intensity and for urine odor intensity. A code was considered to achieve urine odor elimination if it received a urine odor intensity ranking the same or better than the water control.

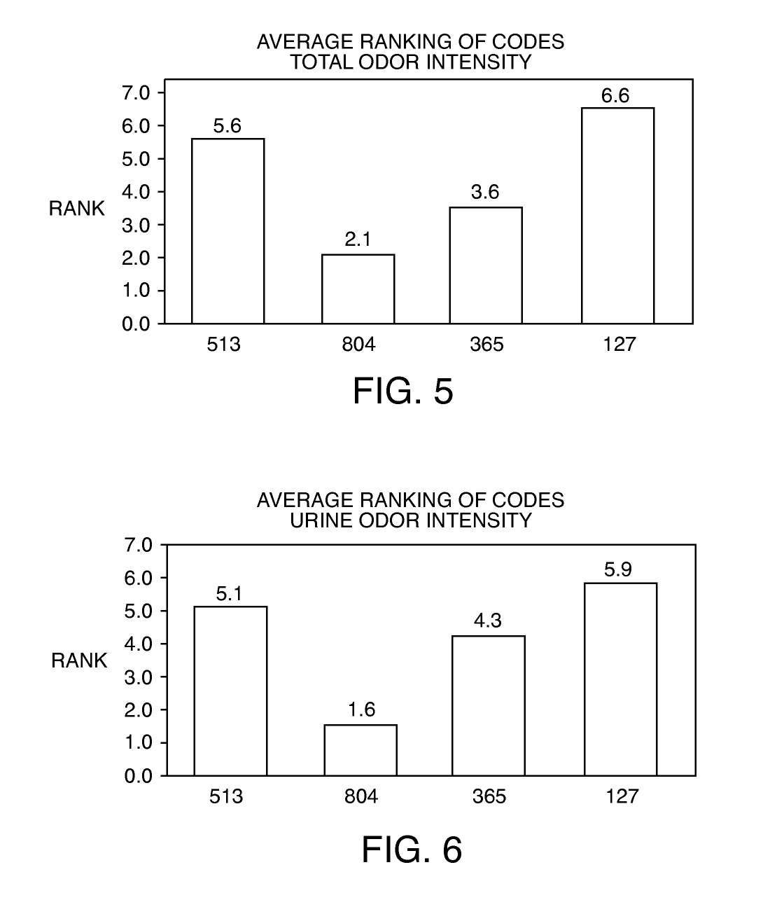

Results: The results are shown in FIGS. 5 and 6 for total odor intensity and for urine odor intensity, respectively, in which the controls were the pad insulted with urine and the pad insulted with water. Codes with rankings above the urine control result were judged to produce fewer odors.

### Example 2

Code ID 874 (Water Control): 10″ by 17″ absorbent-core handsheets were formed by combining 40 grams of BASF HYSORB 8760AD superabsorbent particles with 48.8 grams of WEYERHAEUSER NB416 fluff in a vacuum handsheet former having a tissue-lined screen. The handsheets were densified to the desired thickness of about 6 mm by feeding them through two smooth calender nip rolls at a pressure of about 400 psi.

An oval shaped die about 2.5″ wide by about 9.5″ long was used to cut absorbent cores from the handsheets. The oval-shaped die used to cut the handsheets was also used to cut the tissue. The die-cut tissue was aligned with and placed on both the body-facing surface and the opposite garment-facing surface of the absorbent core to form an absorbent body. The absorbent body was placed in the pad and stapled in place at one end of the pad.

Code ID 106 (Urine Control): The absorbent core and tissue layers described for code ID 874 were also used for this code. As with code ID 874, the die-cut tissue was aligned with and placed on both the body-facing surface and the opposite garment-facing surface of the absorbent core to form an absorbent body. The absorbent body was placed in the pad and stapled in place at one end of the pad.

Code ID 763: Multiple 10″ by 17″ handsheets that contained 11.6 grams of WEYERHAEUSER NB416 fluff were made on a vacuum former having a tissue-lined screen. The handsheets were cut to 10″ by 13″ and sprayed on each side with an aqueous silver formulation, specifically, SILVAGARD M98 containing 2500 ppm silver using a 8001 spray nozzle at a cycle velocity of 800 (80 feet/min), resulting in a spray head pressure of 80 psi. The silver-treated sheets were dried in an air drier for 3 minutes at 95° C. The wet pickup was 297%, indicating that there was about 7,423 ppm of silver on the sheet.

Next, 48.8 gram batches of the silver-treated fluff were mixed with 40.0 gram batches of BASF HYSORB 8760AD superabsorbent particles to make absorbent core handsheets in a vacuum former having a tissue-lined screen. Finally, the absorbent core handsheets were densified to the desired thickness of about 6 mm by feeding through two smooth calendered nip rolls at a pressure of about 400 psi.

An oval shaped die about 2.5″ wide by about 9.5″ long was used to cut absorbent core specimens from the silver-treated handsheet. The oval-shaped die was also used to cut tissue that contained activated carbon. The die-cut activated carbon tissue was aligned with and placed on both the body-facing surface and the opposite garment-facing surface of the absorbent core to form an absorbent body. The absorbent body was placed in the pad and stapled in place at one end of the pad.

Code ID 235: Multiple 10″ by 17″ handsheets that contained 11.6 grams of WEYERHAEUSER NB416 fluff were made on a vacuum former having a tissue-lined screen. The handsheets were cut to 10″ by 13″ and sprayed on each side with an aqueous silver formulation, specifically, BIO-GATE ZSM-MICROSILVER that had been diluted with distilled water to contain 2500 ppm silver. The 8001 spray nozzle was used at a cycle velocity of 800 (80 feet/min), resulting in a spray head pressure of 80 psi. The silver-treated sheets were dried in an air drier for 3 minutes at 95° C. The wet pickup was 294%, indicating that there was about 7,361 ppm of silver on the sheet.

Next, 48.8 gram batches of the silver-treated fluff were mixed with 40.0 gram batches of BASF HYSORB 8760AD superabsorbent particles to make absorbent core handsheets in a vacuum former having a tissue-lined screen. Finally, the absorbent core handsheets were densified to the desired thickness of about 6 mm by feeding through two smooth calendered nip rolls at a pressure of about 400 psi.

An oval shaped die about 2.5″ wide by about 9.5″ long was used to cut absorbent core specimens from the silver-treated handsheet. The oval-shaped die was also used to cut tissue that contained activated carbon. The die-cut activated carbon tissue was aligned with and placed on both the body-facing surface and the opposite garment-facing surface of the absorbent core to form an absorbent body. The absorbent body was placed in the pad and stapled in place at one end of the pad.

CODE 498: 10″ by 17″ absorbent-core handsheets were formed by combining 40 grams of BASF HYSORB 8760AD superabsorbent particles with 48.8 grams of WEYERHAEUSER NB416 fluff in a vacuum handsheet former having a tissue-lined screen. The handsheets were densified to the desired thickness of about 6 mm by feeding them through two smooth calender nip rolls at a pressure of about 400 psi.

An oval shaped die about 2.5″ wide by about 9.5″ long was used to cut absorbent cores from the handsheets. The oval-shaped die used to cut the handsheets was also used to cut the tissue that contained activated carbon. The die-cut activated carbon tissue was aligned with and placed on both the body-facing surface and the opposite garment-facing surface of the absorbent core to form an absorbent body. The absorbent body was placed in the pad and stapled in place at one end of the pad.

The five codes of Example 2 are summarized in Table 2.

TABLE 2

| Code ID | Codes | Components |
|---|---|---|
| 874 | POISE control (water insult) | POISE moderate absorbency pad - untreated |
| 106 | POISE control (urine insult) | POISE moderate absorbency pad - untreated |
| 763 | MWV NUCHAR SA-20 | POISE moderate absorbency pad with activated carbon |
| | Activated Carbon and | treated tissue and SILVAGARD M98 silver treated fluff |
| | SILVAGARD M98 silver | 7423 ppm Ag) |
| | particles | |
| 235 | MWV NUCHAR SA-20 | POISE moderate absorbency pad with activated carbon |
| | Activated Carbon and BIO- | treated tissue and BIO-GATE ZSM MICROSILVER silver |
| | GATE ZSM MICROSILVER | treated fluff (7361 ppm Ag) |
| | silver particles | |
| 498 | MWV NUCHAR SA-20 | POISE moderate absorbency pad with activated carbon |
| | Activated Carbon | treated tissue |

There were 10 pads per Code ID evaluated in the Odor Ranking Panel test.

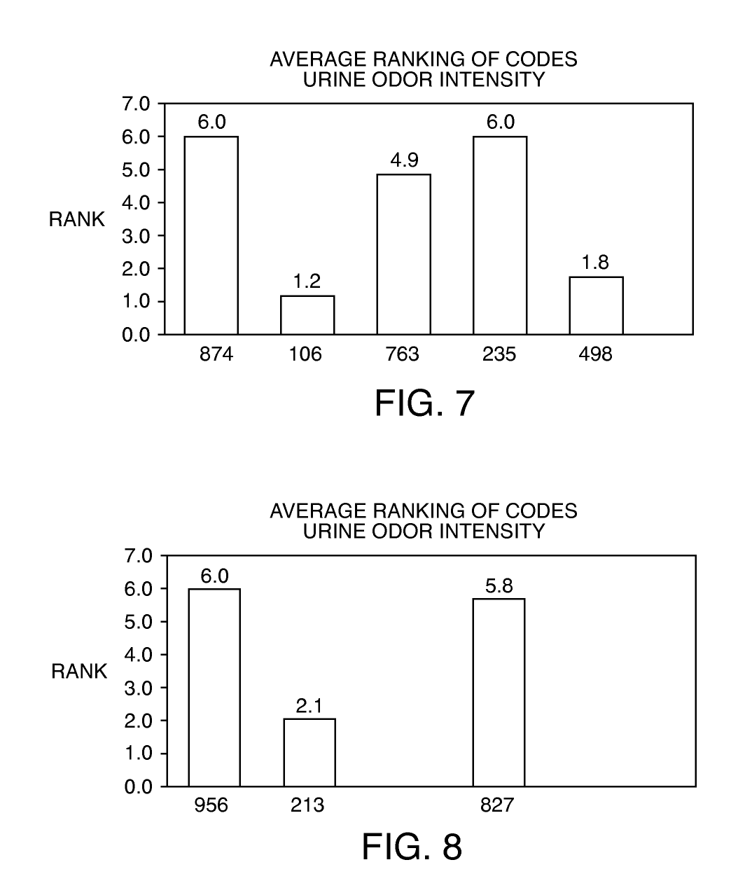

Human urine was collected, pooled, filter sterilized, and then inoculated with bacteria (Proteus mirabilis, Klebsiella pnuemonae, E. faecalis, and E. coli). A fixed amount of urine (78 ml) was placed on each pad and the pad was incubated at 37° C. for four hours. Ten trained panelists were then exposed to each of the codes and asked to rank them for total odor intensity and for urine odor intensity. The results for urine odor intensity are shown in FIG. 7 in which the controls were the pad insulted with urine (code 106) and the pad insulted with water (code 874). Codes with rankings above the urine control result were judged to produce fewer odors.

As can be seen, Code ID 235 was ranked for urine odor intensity the same as the water control, Code ID 874. Therefore, this code with activated carbon in the tissue layer and silver in the fluff of the absorbent core has demonstrated urine odor elimination.

### Example 3

Code ID 956 (Water Control): Multiple 10″ by 17″ absorbent core handsheets that each contained 48.8 grams of WEYERHAEUSER NB416 fluff and 40.0 grams of BASF HYSORB 8760AD superabsorbent particles were made in a vacuum former having a tissue-lined screen. The absorbent core handsheets were densified to the desired thickness of about 6 mm by feeding them through two smooth calendered nip rolls at a pressure of about 400 psi.

An oval shaped die about 2.5″ wide by about 9.5″ long was used to cut absorbent cores from the handsheets. The oval-shaped die used to cut the handsheets was also used to cut the tissue. The die-cut tissue was aligned with and placed on both the body-facing surface and the opposite garment-facing surface of the absorbent core to form an absorbent body. The absorbent body was placed in the pad and stapled in place at one end of the pad.

Code ID 213 (Urine Control): The absorbent core and tissue layers described for code ID 956 were also used for this code. As with code ID 956, the die-cut tissue was aligned with and placed on both the body-facing surface and the opposite garment-facing surface of the absorbent core to form an absorbent body. The absorbent body was placed in the pad and stapled in place at one end of the pad.

Code ID 827: Multiple 10″ by 17″ handsheets that contained 11.6 grams of WEYERHAEUSER NB416 fluff were made on a vacuum former having a tissue-lined screen. The handsheets with WEYERHAEUSER NB416 fluff were cut to 10″ by 13″ and treated by spraying both sides of the sheet with an aqueous silver formulation, specifically, SILVAGARD M98 containing 2500 ppm silver. The treated sheets were dried in an air drier for 3 minutes at 96° C. The wet pickup was 318%, indicating that there was about 7,960 ppm of silver on the sheet.

Next, 48.8 gram batches of the silver-treated fluff were mixed with 40.0 gram batches of BASF HYSORB 8760AD-H superabsorbent particles to make absorbent core handsheets in a vacuum former having a tissue-lined screen. Finally, the absorbent core handsheets were densified to the desired thickness of about 6 mm by feeding them through two smooth calendered nip rolls at a pressure of about 400 psi.

Rectangular shaped pieces about 2.5″ wide by about 9.5″ long were cut from the silver-treated absorbent core handsheets. Tissue containing activated carbon was also cut to this size and aligned with and placed on both the body-facing surface and the opposite garment-facing surface of the absorbent core to form an absorbent body. The absorbent body was placed in the pad and stapled in place at one end of the pad.

The three codes of Example 3 are summarized in Table 3.

TABLE 3

| Code ID | Codes | Components |
|---|---|---|
| 956 | POISE control (water insult) | POISE moderate absorbency pad - untreated |
| 213 | POISE control (urine insult) | POISE moderate absorbency pad - untreated |
| 827 | MWV NUCHAR SA-20 | POISE moderate absorbency pad with activated carbon |
| | Activated Carbon and | treated tissue and with SILVAGARD silver treated fluff |
| | SILVAGARD silver particles | (7960 ppm Ag) |

There were 10 pads per Code ID evaluated in the Odor Ranking Panel test.

Human urine was collected, pooled, filter sterilized, and then inoculated with bacteria (Proteus mirabilis, Klebsiella pnuemonae, E. faecalis, and E. coli). A fixed amount of urine (78 ml) was placed on each pad and the pad was incubated at 37° C. for four hours. Ten trained panelists were then exposed to each of the codes and asked to rank them for total odor intensity and for urine odor intensity.

Results: The results are shown in FIG. 8 for urine odor intensity, in which the controls were the pad insulted with urine (213) and the pad insulted with water (956). Codes with rankings above the urine control result were judged to produce fewer odors. As can be seen, Code ID 827 was judged to have about the same amount of urine odor intensity as the water control, Code ID 956. Therefore, this code with activated carbon in the tissue layer and silver in the fluff of the absorbent core has demonstrated urine odor elimination.

### Example 4

Code ID 293 (Water Control): 10″ by 17″ absorbent-core handsheets were formed by combining 40 grams of BASF HYSORB 8760AD superabsorbent particles with 48.8 grams of WEYERHAEUSER NB416 fluff in a vacuum handsheet former having a tissue-lined screen. The handsheets were densified to the desired thickness of about 6 mm by feeding them through two smooth calender nip rolls at a pressure of about 500 psi.

An oval shaped die about 2.5″ wide by about 9.5″ long was used to cut absorbent cores from the handsheets. The oval-shaped die used to cut the handsheets was also used to cut the tissue. The die-cut tissue was aligned with and placed on both the body-facing surface and the opposite garment-facing surface of the absorbent core to form an absorbent body. The absorbent body was placed in the pad and stapled in place at one end of the pad.

Code ID 645 (Urine Control): The absorbent core and tissue layers described for code ID 293 were also used for this code. As with code ID 293, the die-cut tissue was aligned with and placed on both the body-facing surface and the opposite garment-facing surface of the absorbent core to form an absorbent body. The absorbent body was placed in the pad and stapled in place at one end of the pad.

Code ID 382: Multiple 10″ by 17″ handsheets that contained 11.6 grams of WEYERHAEUSER NB416 fluff were made on a vacuum former having a tissue-lined screen. The handsheets were cut to 10″ by 13″ and treated by spraying both sides of the sheet with an aqueous silver formulation, BIO-GATE ZSM-MICROSILVER that had been diluted with distilled water to contain 690 ppm silver. The treated sheets were dried in an air drier for 4 minutes at 96° C. The wet pickup was 370%, indicating that there was about 2,549 ppm of silver on the sheet.

Next, 48.8 gram batches of fluff were mixed with 40.0 gram batches of BASF HYSORB 8760AD superabsorbent particles to make absorbent core handsheets in a vacuum former having a tissue-lined screen. Finally, the absorbent core handsheets were densified to the desired thickness of about 6 mm by feeding through two smooth calendered nip rolls at a pressure of about 500 psi.

An oval shaped die about 2.5″ wide by about 9.5″ long was used to cut absorbent core specimens from the silver-treated handsheet. The oval-shaped die was also used to cut tissue that contained activated carbon. The die-cut activated carbon tissue was aligned with and placed on both the body-facing surface and the opposite garment-facing surface of the absorbent core to form an absorbent body. The absorbent body was placed in the pad and stapled in place at one end of the pad.

Code ID 426: 10″ by 17″ absorbent-core handsheets were formed as follows. 40.0 g of BASF HYSORB 8760AD superabsorbent particles were mixed with 48.6 g of WEYERHAEUSER NB416 fluff and 0.24 grams of activated carbon (CALGON OL 20×50) in a vacuum former having a tissue-lined screen. Next, the handsheets were densified to the desired thickness of about 6 mm by feeding through two smooth calendered nip rolls at a pressure of about 500 psi.

10″×14″ sheets of 20 gsm plain tissue (without carbon) were individually sprayed with an aqueous silver formulation, specifically, BIO-GATE ZSM-MICROSILVER that had been diluted with distilled water to contain 2971 ppm silver. Each sheet was sprayed twice on one side using an 8001 spray nozzle at a cycle velocity of 630 (63 feet/min), resulting in a spray head pressure of 80 psi. The treated sheets were dried in an air drier for 2.5 minutes at 100° C. The wet pickup was 925%, indicating that there was about 27,484 ppm of silver on the sheet.

An oval shaped die about 2.5″ wide by about 9.5″ long was used to cut absorbent core specimens from the absorbent-core handsheets that contained activated carbon. The oval-shaped die was also used to cut the silver-treated tissue. The die-cut tissue was aligned with and placed on both the body-facing surface and the opposite garment-facing surface of the absorbent core to form an absorbent body. The absorbent body was placed in the pad and stapled in place at one end of the pad.

CODE ID 751: Multiple 10″ by 17″ handsheets that contained 11.6 grams of WEYERHAEUSER NB416 fluff were made on a vacuum former having a tissue-lined screen. The handsheets were cut to 10″ by 13″ and treated by spraying both sides of the sheet with an aqueous silver formulation, BIO-GATE ZSM-MICROSILVER that had been diluted with distilled water to contain 69 ppm silver. The treated sheets were dried in an air drier for 4 minutes at 100° C. The wet pickup was 360%, indicating that there was about 248 ppm of silver on the sheet.

Next, 48.8 gram batches of fluff were mixed with 40.0 gram batches of BASF HYSORB 8760AD superabsorbent particles to make absorbent core handsheets in a vacuum former having a tissue-lined screen. Finally, the absorbent core handsheets were densified to the desired thickness of about 6 mm by feeding through two smooth calendered nip rolls at a pressure of about 500 psi.

An oval shaped die about 2.5″ wide by about 9.5″ long was used to cut absorbent core specimens from the silver-treated handsheet. The oval-shaped die was also used to cut tissue that contained activated carbon. The die-cut activated carbon tissue was aligned with and placed on both the body-facing surface and the opposite garment-facing surface of the absorbent core to form an absorbent body. The absorbent body was placed in the pad and stapled in place at one end of the pad.

Code ID 109: 10″ by 17″ absorbent-core handsheets were formed as follows. 40.0 g of BASF HYSORB 8760AD superabsorbent particles were mixed with 48.6 g of WEYERHAEUSER NB416 fluff and 0.24 grams of activated carbon (CALGON OL 20×50) in a vacuum former having a tissue-lined screen. Next, the handsheets were densified to the desired thickness of about 6 mm by feeding through two smooth calendered nip rolls at a pressure of about 500 psi.

10″×14″ sheets of 20 gsm plain tissue (without carbon) were individually sprayed with an aqueous silver formulation, specifically, BIO-GATE ZSM-MICROSILVER that had been diluted with distilled water to contain 279 ppm silver. Each sheet was sprayed twice on one side using an 8001 spray nozzle at a cycle velocity of 630 (63 feet/min), resulting in a spray head pressure of 80 psi. The treated sheets were dried in an air drier for 3 minutes at 100° C. The wet pickup was 893%, indicating that there was about 2,492 ppm of silver on the sheet.

An oval shaped die about 2.5″ wide by about 9.5″ long was used to cut absorbent core specimens from the absorbent-core handsheets that contained activated carbon. The oval-shaped die was also used to cut the silver-treated tissue. The die-cut tissue was aligned with and placed on both the body-facing surface and the opposite garment-facing surface of the absorbent core to form an absorbent body. The absorbent body was placed in the pad and stapled in place at one end of the pad.

The six codes of Example 4 are summarized in Table 4.

TABLE 4

| Code ID | Codes | Components |
|---|---|---|
| 293 | POISE control (water insult) | POISE moderate absorbency pad - untreated |
| 645 | POISE control (urine insult) | POISE moderate absorbency pad - untreated |
| 382 | MWV NUCHAR SA-20 | POISE moderate absorbency pad with activated carbon |
| | Activated Carbon and BIO- | treated tissue (0.028 g carbon in product) and BIO-GATE |
| | GATE ZSM MICROSILVER | ZSM MicroSilver silver treated fluff (2549 ppm Ag => |
| | silver particles | 0.0144 g Ag in product) |
| 426 | CALGON 20 × 50 Activated | POISE moderate absorbency pad with activated carbon |
| | Carbon and BIO-GATE | treated fluff (at 0.5% => 0.027 g carbon in product) and |
| | ZSM MICROSILVER silver | with silver treated tissue wrap (27,484 ppm Ag => 0.0132 g |
| | particles | Ag in product) |
| 751 | MWV NUCHAR SA-20 | POISE moderate absorbency pad with activated carbon |
| | Activated Carbon and BIO- | treated tissue (0.028 g carbon in product) and BIO-GATE |
| | GATE ZSM MICROSILVER | ZSM MicroSilver silver treated fluff (248 ppm Ag => |
| | silver particles | 0.0013 g Ag in product) |
| 109 | CALGON 20 × 50 Activated | POISE moderate absorbency pad with activated carbon |
| | Carbon and BIO-GATE | treated fluff (at 0.5% => 0.026 g carbon in product) and |
| | ZSM MICROSILVER silver | with silver treated tissue wrap (2,492 ppm Ag => 0.0011 g |
| | particles | Ag in product) |

There were 10 pads per Code ID evaluated in the Odor Ranking Panel test.

Human urine was collected, pooled, filter sterilized, and then inoculated with bacteria (Proteus mirabilis, Klebsiella pnuemonae, E. faecalis, and E. coli). A fixed amount of urine (78 ml) was placed on each pad and the pad was incubated at 37° C. for four hours. Ten trained panelists were then exposed to each of the codes and asked to rank them for total odor intensity and for urine odor intensity.

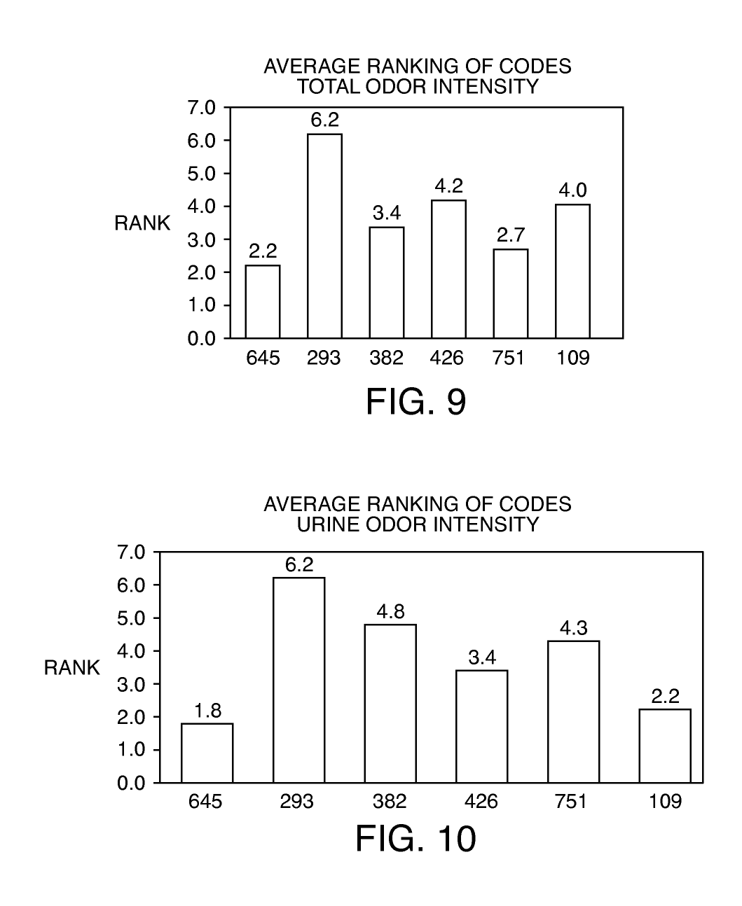

Results: The results are shown in FIGS. 9 and 10 for total odor intensity and for urine odor intensity, respectively, in which the controls were the pad insulted with urine (code 645) and the pad insulted with water (code 293). It can be seen in FIG. 10 that the codes with silver in the fluff and carbon in the tissue (code ID 382 and code ID 751) were more effective at reducing urine odor intensity than the corresponding codes with silver in the tissue and carbon in the fluff (code ID 426 and code ID 109). Also, it is useful to point out in Table 4 that the levels of silver and carbon in the product were adjusted to be the same for code ID 382 versus code ID 426 (high silver codes) and for code ID 751 versus code ID 109 (low silver codes). Therefore, urine odor intensity is due to the placement of the silver and carbon in the product and is lower when silver is placed in the fluff and carbon is placed in the tissue.

As will be appreciated by those skilled in the art, changes and variations to the present disclosure are considered to be within the ability of those skilled in the art. Examples of such changes are contained in the patents identified above, each of which is incorporated herein by reference in its entirety to the extent it is consistent with this specification. Such changes and variations are intended by the inventors to be within the scope of the present disclosure. It is also to be understood that the scope of the present disclosure is not to be interpreted as limited to the specific aspects disclosed herein, but only in accordance with the appended claims when read in light of the foregoing disclosure.

1. A personal care product comprising:
an absorbent core covered by a substrate;
wherein the absorbent core comprises silver particles, and the substrate comprises activated carbon.

2. The personal care product of claim 1, wherein the personal care product is selected from the group consisting of: a diaper, a training pant, an absorbent underpant, an adult incontinence product, and a feminine hygiene product.

3. The personal care product of claim 1, wherein the activated carbon is in the form of particles that are less than 100 microns in diameter.

4. The personal care product of claim 1, wherein the activated carbon is in an amount of 0.01g to 0.40 g.

5. The personal care product of claim 1, wherein the silver particles are not associated with a binder.

6. The personal care product of claim 1, wherein the silver particles are nanoparticles.

7. The personal care product of claim 1, wherein the silver particles are microparticles in an amount of 0.0001 g to 0.05 g.

8. The personal care product of claim 1, wherein the silver particles are microparticles in an amount of 0.001 g to 0.02 g.

9. The personal care product of claim 1, further comprising
a bodyside liner; and
an outer cover;
wherein the absorbent core and the substrate are disposed between the bodyside liner and the outer cover.

10. The personal care product of claim 1, wherein the substrate is tissue.

11. The personal care product of claim 1, wherein the substrate is directly adjacent to the absorbent core.

12. The personal care product of claim 1, wherein the substrate completely covers a body-facing surface of the absorbent core.

13. The personal care product of claim 1, further comprising a surge member disposed between the substrate and the absorbent core.

14. A method of reducing urine odor in a personal care product to a level below human detection, the method comprising the steps of:
disposing a substrate comprising activated carbon onto an absorbent core comprising silver particles; and
disposing the substrate and absorbent core between an outer cover and a bodyside liner.

15. The method of claim 14, wherein the silver particles do not migrate through the substrate.

16. The method of claim 14, wherein the silver particles are uniformly dispersed throughout the absorbent core.

17. The method of claim 14, wherein the absorbent core has a body-facing surface, a garment-facing surface, and longitudinal sides, and wherein the substrate is disposed onto the absorbent core by wrapping it around the body-facing surface and longitudinal sides.

18. The method of claim 14, wherein the absorbent core has a body-facing surface, a garment-facing surface, and longitudinal sides, and wherein the substrate is a strip that is disposed onto the garment-facing surface of the absorbent core.

19. The method of claim 14, wherein the absorbent core has a body-facing surface, a garment-facing surface, and longitudinal sides, and wherein the substrate is a strip that is disposed onto the body-facing surface of the absorbent core.

20. The personal care product of claim 1, wherein the substrate does not include silver particles.
